(19) United States
(12) Patent Application Publication
(10) Pub. No.: US 2026/0256465 A1
(43) Pub. Date: Sep. 3, 2026

(54) Open Tendon Stripper

(71) Applicant: Conmed Corporation, Largo, FL (US)

(72) Inventor: Andrew Kam, Odessa, FL (US)

(73) Assignee: Conmed Corporation, Largo, FL (US)

(21) Appl. No.: 19/555,387

(22) Filed: Mar. 3, 2026

Related U.S. Application Data
(60) Provisional application No. 63/765,996, filed on Mar. 3, 2025.

(51) Int. Cl. A61B 17/00

(52) U.S. Cl. CPC A61B 17/00008; A61B 2017/00349

(57) ABSTRACT

An open tendon stripper configured and structured to enable a medical practitioner user to selectively harvest a portion of the tendon's thickness, while preserving native tendon, is disclosed. In accordance with an embodiment, a quad tendon stripping device can include an elongated shaft having a distal end and a proximal end, where the proximal end is connected to a handle and the distal end is connected to a hook-shaped tip having a frame with a base width and an opening width. In accordance with an embodiment, the base width is greater than the opening width.

### BACKGROUND

### 1. Field

The present disclosure relates generally to a medical/surgical cutting device, and more particularly to a cutting device that is structured and configured to facilitate minimally invasive harvesting of the quadriceps tendon for use as a graft for ligament reconstruction.

### 2. Description of the Related Art

Surgical reconstruction of the knee often requires the harvesting of soft-tissue grafts, such as the quadriceps tendon. As a result, tools have been developed to facilitate the efficient and safe harvesting of quad tendon tissue.

Description of the Related Art Section Disclaimer: To the extent that specific patents/publications/products are discussed above in this Background Section or elsewhere in this Application, these discussions should not be taken as an admission that the discussed patents/publications/products are prior art for patent law purposes. For example, some or all of the discussed patents/publications/products may not be sufficiently early in time, may not reflect subject matter developed early enough in time and/or may not be sufficiently enabling so as to amount to prior art for patent law purposes. To the extent that specific patents/publications/products are discussed above in this Background Section and/or throughout the application, the descriptions/disclosures of which are all hereby incorporated by reference into this document in their respective entirety(ies).

### BRIEF SUMMARY

The inventors of the present disclosure recognize that there are problems and limitations with conventional quad tendon harvesting devices and related methods/processes of use. Traditional graft harvesting tools often require extensive surgical exposure and do not accommodate for varying tendon thicknesses. For instance, when the quad tendon is harvested to reconstruct a ligament, a certain graft size (e.g., thickness) is desired depending on the patient's anatomy and the type of reconstruction. To achieve the desired graft size, it may necessitate harvesting only a portion of the tendon, such as two of the three layers (i.e., superficial, intermediate, and deep). Therefore, there is a need and it would be beneficial to have a device that is able to harvest only the necessary portions of a tendon or other tissue while leaving the remaining layers intact and preserving the native tissue.

It is therefore a principal object and advantage of the present disclosure to provide an open tendon stripper that is configured and structured to enable a medical practitioner user to selectively harvest a portion of the tendon's thickness, while preserving native tendon.

In accordance with an embodiment, a quad tendon stripping device is provided. The quad tendon stripper can include an elongated shaft having a distal end and a proximal end, where the proximal end is connected to a handle and the distal end is connected to a hook-shaped tip having a frame with a base width and an opening width.

In accordance with an embodiment, the base width can be greater than the opening width. In an alternative embodiment, the base width can be less than the opening width or can also be the same as the opening width.

In accordance with an embodiment, the hook-shaped tip includes a first portion, a second portion, and a third portion, wherein the first and third portions extend in the Y-direction and the second portion extends in the Z-direction.

In accordance with an embodiment, a free end of the hook-shaped tip of the tendon stripper device can be substantially level with a bottom surface of the elongated shaft.

In accordance with an embodiment, the hook-shaped tip further includes a leading edge and a trailing edge, wherein the leading and trailing edges are configured to separate tendon fibers.

In accordance with an embodiment, the elongated shaft of the tendon stripper device further includes measurement markings etched therein.

In accordance with an embodiment, the hook-shaped tip and the handle are integrally formed (e.g., formed from a single piece of material, or a single mold) with the elongated shaft of the tendon stripper device.

In accordance with an embodiment, the hook-shaped tip and the handle are connected to the elongated shaft via friction fit, soldier or other connection process/mechanism (as should be understood by those of ordinary skill in the art in conjunction with a review of this disclosure).

In accordance with an embodiment, the hook-shaped tip is positioned immediately adjacent to a free end of the elongated shaft. In an alternative embodiment, the hook-shaped tip is positioned a predetermined distance from the free end of the elongated shaft, but is closer to the free end of the elongated shaft than to the middle of the shaft.

In accordance with another embodiment, a method of stripping a tendon is described. A tendon stripping device is provided that comprises an elongated shaft having a distal end and a proximal end, where the proximal end is connected to a handle and the distal end is connected to a hook-shaped tip having a frame with a base width and an opening width. The stripping device can have one or more of the components and/or functionalities as described herein.

In accordance with an embodiment, the base width is greater than the opening width.

In accordance with an embodiment, the hook-shaped tip comprises a first portion, a second portion, and a third portion, wherein the first and third portions extend in the Y-direction and the second portion extends in the Z-direction.

In accordance with an embodiment, a free end of the hook-shaped tip of the tendon stripper device is substantially level with a bottom surface of the elongated shaft.

In accordance with an embodiment, the hook-shaped tip further comprises a leading edge and a trailing edge, wherein the leading and trailing edges are configured to separate tendon fibers.

In accordance with an embodiment, the elongated shaft of the tendon stripper device further comprises measurement markings etched therein (and placed thereon as should be understood by a person of ordinary skill in the art in conjunction with a review of this disclosure).

In accordance with an embodiment, the hook-shaped tip and the handle are integrally formed with the elongated shaft of the tendon stripper device.

In accordance with an embodiment, the hook-shaped tip and the handle are connected to the elongated shaft via friction fit.

In accordance with an embodiment, the hook-shaped tip is positioned immediately adjacent to a free end of the elongated shaft.

In accordance with an embodiment, the method further includes the step of manipulating the handle (clockwise, counterclockwise about the central longitudinal axis thereof, forward and/or backwards in the direction of the longitudinal axis) to move the leading and trailing edges distally and proximally along a length of the tendon.

### BRIEF DESCRIPTION OF THE DRAWINGS

Embodiments of the present disclosure will be more fully understood and appreciated by reading the following Detailed Description in conjunction with the accompanying drawings. Various embodiments of the present disclosure may be advantageous in that they may solve or reduce one or more of the potential problems and/or disadvantages discussed herein. The accompanying drawings illustrate only typical embodiments of the disclosed subject matter and are therefore not to be considered limiting of its scope, for the disclosed subject matter may admit to other equally effective embodiments.

Reference is now made briefly to the accompanying drawings, in which:

FIG. 1 is a close-up view of a quad tendon.

FIG. 2 is a perspective view of the quad tendon stripper in accordance with an embodiment of the present disclosure.

FIG. 3 is a close-up view of the distal end of the quad tendon stripper capturing a tendon segment for illustrative purposes in accordance with an embodiment of the present disclosure.

FIG. 4 is a side view schematic representation of the distal end of the quad tendon stripper in accordance with an embodiment of the present disclosure.

FIG. 5A is a front view of the distal end of the quad tendon stripper in accordance with an embodiment of the present disclosure.

FIG. 5B is a perspective view of the distal end of the quad tendon stripper in accordance with an embodiment of the present disclosure.

### DETAILED DESCRIPTION

Aspects of the present embodiments and certain features, advantages, and details thereof, are explained more fully below with reference to the non-limiting examples illustrated in the accompanying drawings. Descriptions of well-known structures are omitted so as not to unnecessarily obscure embodiments in detail. It should be understood, however, that the detailed description and the specific non-limiting examples, while indicating aspects of the embodiments, are given by way of illustration only, and are not by way of limitation. Various substitutions, modifications, additions, and/or arrangements, within the spirit and/or scope of the underlying inventive concepts will be apparent to those skilled in the art from this disclosure.

While embodiments of the present disclosure have been particularly shown and described with reference to certain exemplary embodiments, it will be understood by one skilled in the art that various changes in detail may be affected therein without departing from the spirit and scope of the invention as defined by claims that can be supported by the written description and drawings. Further, where exemplary embodiments are described with reference to a certain number of elements it will be understood that the exemplary embodiments can be practiced utilizing either less than or more than the certain number of elements. If elements shown in a particular Figure discussed below are not specifically identified with respect to that Figure, the elements should be sufficiently identified with respect to at least one other Figure.

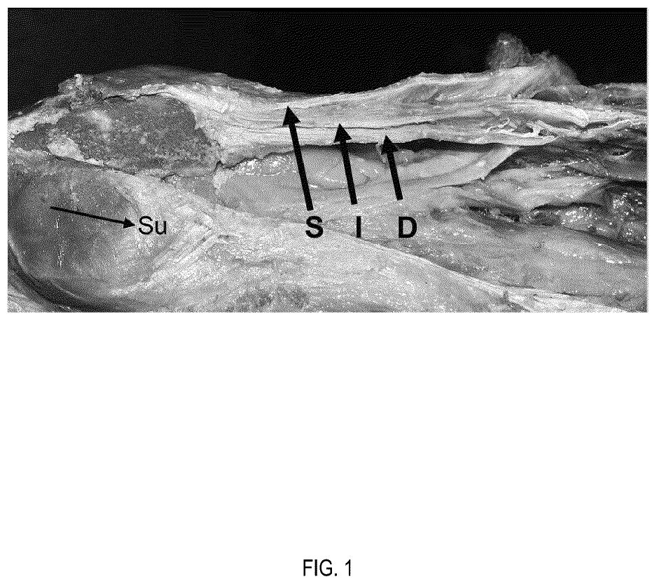

Referring to FIG. 1, a close-up view of a quadriceps tendon is shown for illustrative purposes. The quad tendon is arranged in three layers: superficial (S), intermediate (I), and deep (D).

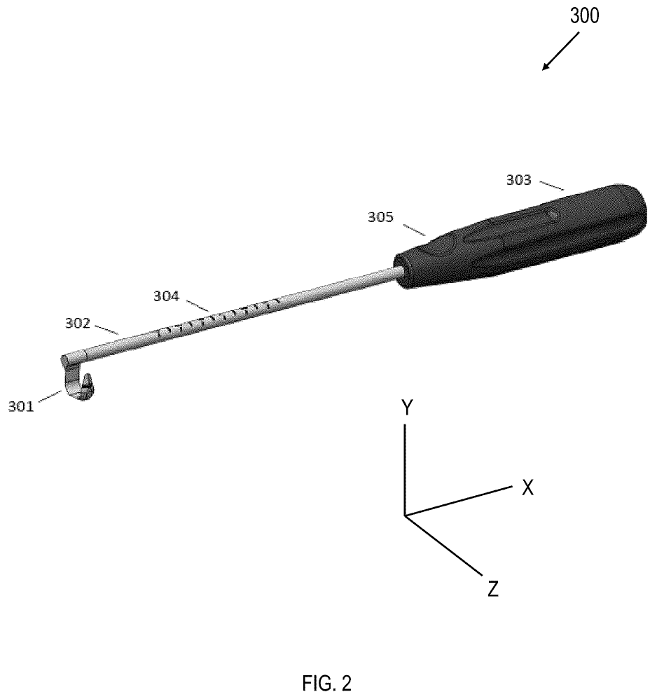

Referring to FIG. 2, a quad tendon stripper 300 is shown according to an embodiment. In this embodiment, the tendon stripper 300 can include a tip 301 attached to a distal end of an elongated shaft 302 and a handle 303 attached to a proximal end of the shaft 302. The attachment of the tip 301 as shown and described with respect to the shaft 302 and handle 303 can provide sufficient access and allow for manual manipulation of the tip 301. In some examples, markings 304 on the shaft (e.g., millimeter, centimeter, and/or inch markings) are included that can provide a reference to how deep the tip 301 is positioned at and within a surgical site (i.e., the markings can begin at the most distal end of the shaft and continue along its length, or begin at any other portion of the shaft). In some embodiments, the markings are etched into the shaft 304, and in other embodiments, the markings are printed or otherwise displayed on the shaft 304. In some embodiments, the handle 303 can have one or more physical features 305 (e.g., divots, carve outs, and/or protrusions) to provide ergonomic and a tactile orientation indication of the tip 301. One or each of the tip 301 and/or the handle 303 can be integrally formed with the shaft 304 (i.e., be one piece) or can be attached thereto via an adhesive, welding, friction fit, etc. (as should be understood by a person of ordinary skill in the art in conjunction with a review of this disclosure). In addition, in some embodiments, the tip 301 can be connected to the most distal end of the shaft 304, immediately adjacent thereto, or can be positioned at a predetermined distance from the most distal end of the shaft 304 but still being closer to the distal end of the shaft 304 than the midpoint of the shaft 304 (or the device as measured by the length of the shaft plus the handle).

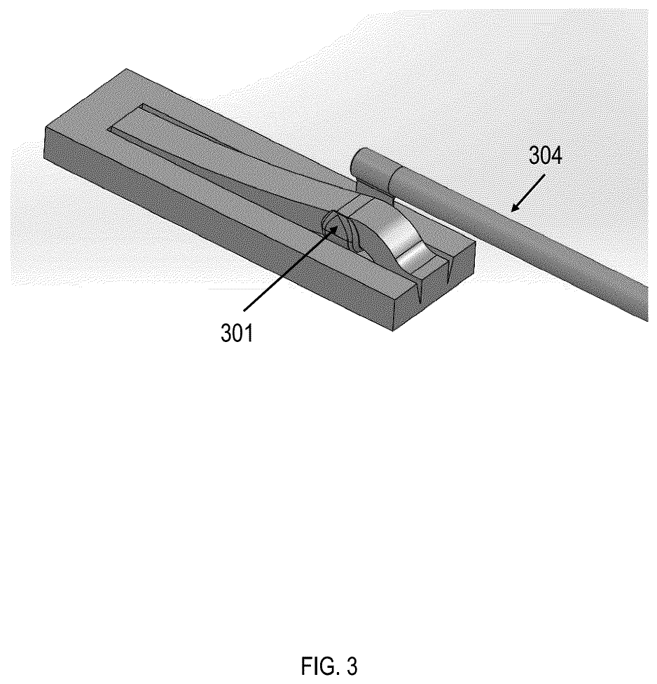

Referring to FIG. 3, the tip 301 of the quad tendon stripper 300 can have an open architecture (i.e., the body of the tip 301 can exhibit a curvature that defines a space where the tendon can be positioned and held during use) which can aid in capturing a tendon while the tendon is still attached to the muscle bony insertion. In a preferred embodiment, the tip 301 is dimensioned and configured to hook around the tendon segment after it has been pre-cut to a specified width and depth. Once the tip 301 is placed around the tendon, the sharpened leading and trailing edges thereof can function to separate the fibers of the tendon proximally and distally, while staying within the desired plane of the tendon. While the leading and trailing edges of the frame can be sharpened, they can be specifically sharpened to aid in separating the tendon fibers, but not sharpened to the degree of a knife blade, to prevent the tip from inadvertently cutting the tendon (in instances where that is not desired, and leading to a graft length that is shorter than desired). In some embodiments, the most distal portion (i.e., free end) of the tip 301 is substantially level (+/−5% of the tip's length) with the bottom surface of the shaft, and in other embodiments, the most distal portion of the tip 301 is positioned in a plane either above or below that of the bottom surface of the shaft 304.

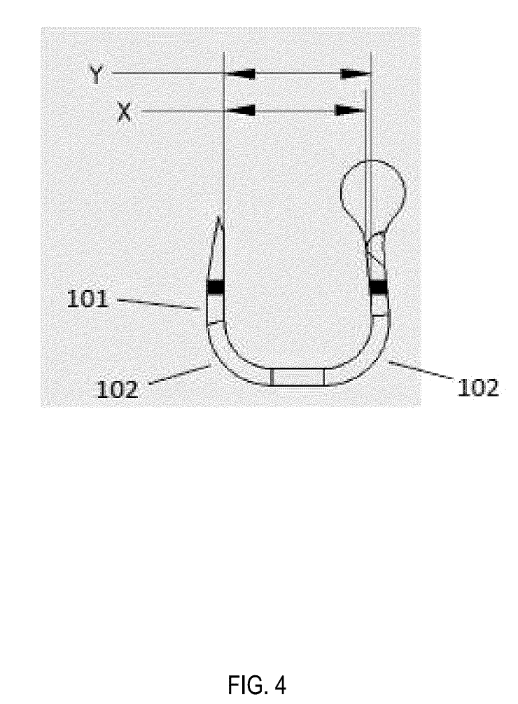

Referring to FIG. 4, a schematic illustration of the tip 301 is shown. In this example, the geometry of the tip 101 can feature a hook or U-shaped open frame to allow for placing the tip around the tendon segment that is intended to be harvested (as shown in FIG. 3). In some embodiments, the frame width can be narrower at the opening X relative to the base Y to aid in keeping the graft captive during the stripping process. In some embodiments, the corners 102 of the frame can be radiused to allow for some rotation of the tip during striping, while preventing damage to surrounding tissue. In a preferred embodiment, the opening X is sized and dimensioned to accept a quad tendon. In the embodiment shown in FIG. 2, the elongated shaft 304 extends in the X direction, while at least a portion of the tip 301 extends in the X direction and at least a portion of the tip 301 extends in the Y direction. Once the tip 301 is positioned underneath a tendon to be stripped, the user can manipulate the handle 303 to move the leading and trailing edges (discussed below) distally and proximally along a length of the tendon to separate it.

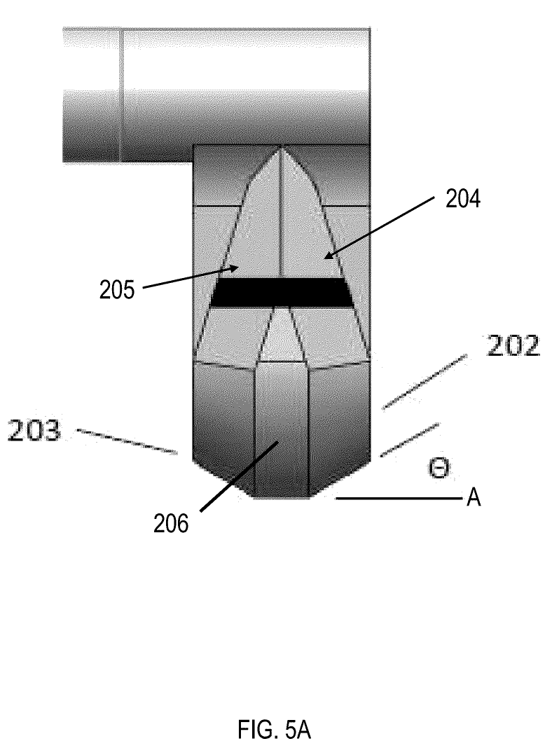

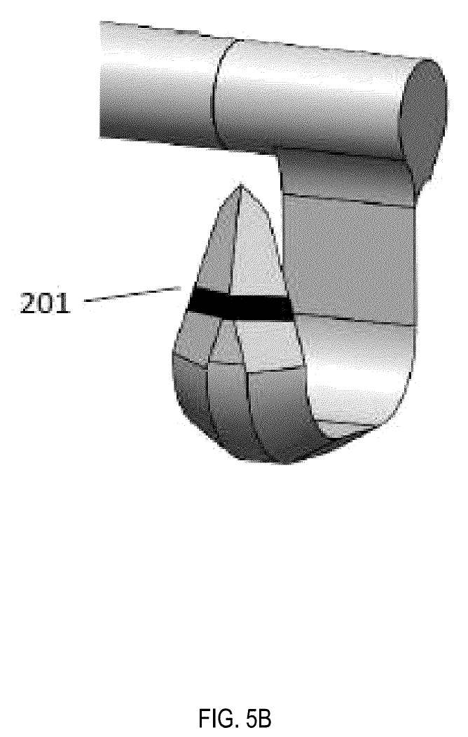

Referring now to FIGS. 5A-5B, as shown, a free leg 201 of the open frame can come to a sharpened point/tip to aid in exposing the tip from the opposite side of the entry location of the graft segment (see, e.g., FIG. 3). One or each of the leading 202 and trailing 203 edges can be sharpened to aid with fiber stripping, for example, but not excessively sharp so as to cut through the tendon graft, controlled by the angle Θ of the edge(s). According to an embodiment, angle Θ can range between about 10°-50° with respect to a bottom axis A of the tip. In a preferred embodiment, angle Θ is about 30° with respect to bottom axis A (see, e.g., FIG. 3A). According to an embodiment, the sharpness of each of the edges shown can be tuned to fit the particular circumstance, keeping in mind that a main purpose of the tip is not to start and finish a cut through the tendon, but to aid in separating the tendon fibers (as discussed above with respect to FIG. 3). In some embodiments, the free leg 201 is made up of a first face 204 and a second face 205, where the faces are positioned at an angle to each other, forming a beveled surface. In an alternative embodiment, the first and second faces 204 and 205 are positioned substantially along the same plane. In accordance with an embodiment, a middle edge 206 is positioned between the leading and trailing edges 202 and 203, wherein at least a portion of the middle edge is substantially level with respect to the bottom axis A (i.e., angle Θ is about 0-10° with respect to A, as shown in FIG. 3A).

In accordance with an embodiment, the shaft 304 and tip 301 can be made of a stainless-steel material, but other similar materials are contemplated. In cases where the material is stainless-steel, the quad tendon stripper 300 can be autoclavable and re-useable. In an alternative embodiment, the components of the quad tendon stripper 300 can be of a durable plastic material and thus intended for one-time use.

It should be understood that any of the specific dimensions recited in this application can apply to any embodiment described above. All embodiments and elements thereof can be combined in any way mechanically possible and are not intended to be limited to its respective embodiment unless otherwise stated.

The terminology used herein is for the purpose of describing particular embodiments only and is not intended to be limiting of the embodiments. As used herein, the singular forms “a”, “an”, and “the” are intended to include the plural forms as well, unless the context clearly indicates otherwise. It will be further understood that the terms “comprise” (and any form of comprise, such as “comprises” and “comprising”), “have” (and any form of have, such as, “has” and “having”), “include” (and any form of include, such as “includes” and “including”), and “contain” (any form of contain, such as “contains” and “containing”) are open-ended linking verbs. As a result, a method or device that “comprises”, “has”, “includes” or “contains” one or more steps or elements. Likewise, a step of method or an element of a device that “comprises”, “has”, “includes” or “contains” one or more features possesses those one or more features but is not limited to possessing only those one or more features. Furthermore, a device or structure that is configured in a certain way is configured in at least that way but may also be configured in ways that are not listed.

The corresponding structures, materials, acts and equivalents of all means or step plus function elements in the claims below, if any, are intended to include any structure, material or act for performing the function in combination with other claimed elements as specifically claimed. The description of the present embodiments has been presented for purposes of illustration and description but is not intended to be exhaustive or limited to the embodiments in the form disclosed. Many modifications and variations will be apparent to those of ordinary skill in the art without departing from the scope and spirit of the embodiments. The embodiment was chosen and described in order to best explain the principles of one or more aspects of the embodiments and the practical application, and to enable others of ordinary skill in the art to understand one or more aspects of the present embodiments for various embodiments with various modifications as are suited to the particular use contemplated.

What is claimed is:

1. A tendon stripper device, comprising:
an elongated shaft having a distal end and a proximal end;
the proximal end being connected to a handle;
the distal end being connected to a hook-shaped tip having a frame with a base width and an opening width, wherein the base width is greater than the opening width; and
wherein the hook-shaped tip comprises a first portion, a second portion, and a third portion, wherein the first and third portions extend substantially in the Y-direction and the second portion substantially extends in the Z-direction.

2. The tendon stripper device of claim 1, wherein a free end of the hook-shaped tip is substantially level with a bottom surface of the elongated shaft.

3. The tendon stripper device of claim 1, wherein the hook-shaped tip further comprises a leading edge and a trailing edge, wherein the leading and trailing edges are configured to separate tendon fibers.

4. The tendon stripper device of claim 1, wherein the elongated shaft further comprises measurement markings etched therein.

5. The tendon stripper device of claim 1, wherein the hook-shaped tip and the handle are integrally formed with the elongated shaft.

6. The tendon stripper device of claim 1, wherein the hook-shaped tip and the handle are connected to the elongated shaft via friction fit.

7. The tendon stripper device of claim 1, wherein the hook-shaped tip is positioned immediately adjacent to a free end of the elongated shaft.

8. A method of stripping a tendon, comprising:
providing a tendon stripping device, comprising:
an elongated shaft having a distal end and a proximal end;
the proximal end being connected to a handle;
the distal end being connected to a hook-shaped tip having a frame with a base width and an opening width, wherein the base width is greater than the opening width; and
wherein the hook-shaped tip comprises a first portion, a second portion, and a third portion, wherein the first and third portions extend in the Y-direction and the second portion extends in the Z-direction;
positioning the hook-shaped tip underneath a tendon segment to be stripped;
applying an upward force on the handle to separate fibers of the tendon.

9. The method of claim 8, wherein a free end of the hook-shaped tip of the tendon stripper device is substantially level with a bottom surface of the elongated shaft.

10. The method of claim 8, wherein the hook-shaped tip of the tendon stripper device further comprises a leading edge and a trailing edge, wherein the leading and trailing edges are configured to separate tendon fibers.

11. The method of claim 8, wherein the elongated shaft of the tendon stripper device further comprises measurement markings etched therein.

12. The method of claim 8, wherein the hook-shaped tip and the handle are integrally formed with the elongated shaft of the tendon stripper device.

13. The method of claim 8, wherein the hook-shaped tip and the handle are connected to the elongated shaft of the tendon stripper device via friction fit.

14. The method of claim 8, wherein the hook-shaped tip is positioned immediately adjacent to a free end of the elongated shaft.

15. The method of claim 8, further comprising the step of manipulating the handle to move the leading and trailing edges distally and proximally along a length of the tendon.
